(19) United States
(12) Patent Application Publication
(10) Pub. No.: US 2016/0206583 A1
(43) Pub. Date: Jul. 21, 2016

(54) AMINO ACID TREATMENT OF SEIZURES

(71) Applicant: THE JOHNS HOPKINS UNIVERSITY, Baltimore, MD (US)

(72) Inventors: ADAM HARTMAN, LUTHERVILLE-TIMONIUM, MD (US); POLAN SANTOS, HANOVER, MD (US); J. MARIE HARDWICK, BALTIMORE, MD (US)

(73) Assignee: THE JOHNS HOPKINS UNIVERSITY, BALTIMORE, MD (US)

(21) Appl. No.: 14/914,774

(22) PCT Filed: Aug. 27, 2014
(86) PCT No.: PCT/US14/52946

§ 371 Date: Feb. 26, 2016

Related U.S. Application Data
(60) Provisional application No. 61/870,539, filed on Aug. 27, 2013.
(60) Provisional application No. 61/940,615, filed on Feb. 17, 2014.

(51) Int. Cl. A61K 31/198; A61K 31/423; A61K 31/5513

(52) U.S. Cl. CPC A61K 31/198; A61K 31/5513; A61K 31/423

(57) ABSTRACT

Methods for treating or preventing a seizure in a subject by administering to the subject a therapeutically effective amount of at least one D-amino acid or D-amino acid oxidase inhibitor are provided. In certain aspects, the method reduces the frequency, severity, and/or duration of one or more seizures in the subject. In particular aspects, at least one D-amino acid is administered to the subject before an onset of a seizure, during a seizure, or after a seizure to prevent further seizures. In certain aspects, at least one D-amino acid is administered to the subject prophylactically to prevent the occurrence of a seizure. In more particular aspects, the seizure is caused by epilepsy.

### CROSS-REFERENCE TO RELATED APPLICATIONS

This application claims the benefit of U.S. Provisional Application No. 61/870,539, filed Aug. 27, 2013, and U.S. Provisional Application No. 61/940,615, filed Feb. 17, 2014. Each of the afore-mentioned applications is incorporated herein by reference in its entirety.

### FEDERALLY SPONSORED RESEARCH OR DEVELOPMENT

This invention was made with government support under 1K08NS070931, K12NS001696, NS083373, and NS037402 awarded by the National Institutes of Health (NIH). The government has certain rights in the invention.

### BACKGROUND

Amino acids are biologically important organic compounds made from amine (—NH₂) and carboxylic acid (—COOH) functional groups, along with a side-chain specific to each type of amino acid. All amino acids (except glycine) can occur in two isomeric forms, because of the possibility of forming two different enantiomers (stereoisomers) around the central carbon atom. By convention, these stereoisomers are referred to as “L-” and “D-” forms, analogous to left-handed and right-handed configurations. Only L-amino acids are manufactured in cells and incorporated into proteins, although a few D-amino acids occur in bacterial envelopes and some antibiotics. The L-enantiomers of amino acids are widely assumed to account for most of their biological effects, including signaling, transporter-mediated protein interactions, and as a metabolic substrate.

With respect to D-amino acids in the brain, D-leucine is present in the hippocampus and pineal gland, for example, in 6 week-old rat pups, but it is not found in appreciable concentrations in the cerebrum, cerebellum, medulla or pituitary gland (Hamase et al., 1997). In contrast, D-serine (a potent ligand for the glycine binding site on the N-methyi-D-aspartate (NMDA) receptor) is found in the cerebrum, the hippocampus, and in the pituitary and pineal glands (Hamase et al., 1997). The only other D-amino acids detected by Hamase et al. were D-aspartic acid (pituitary and pineal glands) and D-alanine (only pituitary gland) (Hamase et al., 1997). In mammals, D-leucine typically comes from dietary sources, including beer (Ekborg-Ott and Armstrong, 1996). Evidence of a role for D-leucine in neurological activity was shown in a study of equine pain treatment, where mention was made (without direct proof) that it binds to enkephalinases (McKibbin and Cheng, 1982).

The brain controls how the body moves by sending out small electrical signals through the nerves to the muscles. Seizures, or convulsions, occur when abnormal signals from the brain change the way the body functions. One underutilized option for patients whose seizures are not controlled by medicines is metabolism-based therapy (Hartman and Stafstrom, 2013). Inhibition of the nutrient-sensing serine-threonine kinase mammalian target of rapamycin (mTOR) pathway has shown promise in preventing the development of seizures (Zeng et al., 2009; Ljungberg et al., 2009). L-leucine (L-Leu) is a well-established activator of mTORC1 (Sancak et al., 2008), and has been reported to activate mTORC1 activity in the brain (Cota et al., 2006 and Hartman et al., unpublished data).

### SUMMARY

In one aspect, the presently disclosed subject matter provides a method for treating or preventing a seizure in a subject, the method comprising administering to the subject a therapeutically effective amount of at least one D-amino acid. In certain aspects, the method reduces the frequency, severity, and/or duration of one or more seizures in the subject.

In particular aspects, at least one D-amino acid is administered to the subject before an onset of a seizure, during a seizure, or after a seizure to prevent further seizures. In certain aspects, at least one D-amino acid is administered to the subject prophylactically to prevent the occurrence of a seizure. In more particular aspects, the seizure is caused by epilepsy.

In another aspect, the presently disclosed subject matter provides a method for treating or preventing a seizure in a subject, the method comprising administering to the subject a therapeutically effective amount of at least one D-amino acid oxidase inhibitor.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIGS. 1A-1B show that D-leucine pretreatment protects against 6 Hz-induced seizures: (A) probability of seizures was determined by a probit analysis. Results presented are for weight-matched mice treated with D-leucine (1.5% w/v) in drinking water or regular untreated water, tested in 3 independent animal cohorts in 3 independent experiments; and (B) the current where 50% of mice had convulsions, where CC50 was derived from data in FIG. 1A (larger animal numbers were tested near the CC50 to increase sensitivity of the assay). Statistically significant values are highlighted in grey;

FIGS. 2A-2B show that D-leucine pretreatment protects against seizures: (A) Mean seizure scores (±SEM) taken at 5-min intervals in the kainic acid (23.5 mg/kg) status epilepticus test for 3-4 independent cohorts of mice pretreated with D-leucine (0.3, 3, or 300 mg/kg) or water (vehicle) for 3 h and then observed for 2 h following kainic acid administration; (B) Table showing seizure outcomes for each treatment group in the kainic acid test. †P<0.0001 (ANOVA), P<0.001 H₂O vs D-leu (300 mg/kg), P<0.01 H₂O vs D-leu (3 mg/kg), D-leu (300 mg/kg) vs D-leu (0.3 mg/kg), P<0.05 D-leu (3 mg/kg) vs D-leu (0.3 mg/kg) (post-hoc Tukey); ††P<0.0001 (ANOVA), P<0.001 H₂O vs D-leu (300 mg/kg), H₂O vs D-leu (3 mg/kg), D-leu (300 mg/kg) vs D-leu (0.3 mg/kg), D-leu (3 mg/kg) vs D-leu 0.3 mg/kg) (post-hoc Tukey); ‡P=0.0004 (ANOVA), P<0.001 H₂O vs D-leu (300 mg/kg), P<0.01 D-leu (300 mg/kg) vs D-leu (0.3 mg/kg) (post-hoc Tukey); ‡‡P=0.02 (ANOVA), P<0.05 H₂O vs D-leu (300 mg/kg) (post-hoc Tukey);

FIGS. 3A-3B show that CBIO pretreatment protects against seizures induced by kainic acid (i.p.): (A) mean seizure scores (±SEM) taken at 5-min intervals for two independent cohorts of mice treated with CBIO (10 mg/kg) for 3 h (NS: control mice not treated with CBIO); and (B) table showing seizure outcomes for each treatment group. Statistically significant values are highlighted in grey;

FIGS. 4A-4D show that D-leucine treatment after seizure onset protects against seizures induced by kainic acid (i.p.): (A) mean seizure scores (±SEM) taken at 5-min intervals for two independent cohorts of mice treated with D-leucine (300 mg/kg) 15 min after the onset of seizures; (B) table showing seizure outcomes for each treatment group. Statistically significant values are highlighted in grey; (C) mean seizure scores (±SEM) taken at 5-min intervals for two independent cohorts of mice treated with D-leucine (3 mg/kg) 15 min after the onset of seizures (arrow); and (D) table showing seizure outcomes for each treatment group;

FIGS. 5A-5B show that CBIO treatment after seizure onset protects against seizures induced by kainic acid (i.p.): (A) mean seizure scores (±SEM) taken at 5-min intervals for two independent cohorts of mice treated with CBIO (10 mg/kg) 15 min after the onset of seizures; and (B) table showing seizure outcomes for each treatment group. Statistically significant values are highlighted in grey;

FIGS. 6A-6B show that D-serine treatment (300 mg/kg) after seizure onset protects against seizures induced by kainic acid (i.p.): (A) mean seizure scores (±SEM) taken at 5-min intervals for one cohort of mice treated with D-serine (300 mg/kg) 15 min after the onset of seizures; and (B) table showing seizure outcomes for each treatment group. Statistically significant values are highlighted in grey;

FIGS. 7A-7B show that D-serine treatment (3 mg/kg) after seizure onset protects against seizures induced by kainic acid (i.p.): (A) mean seizure scores (±SEM) taken at 5-min intervals for 1 cohort of mice treated with D-serine (3 mg/kg) 15 min after the onset of seizures; and (B) table showing seizure outcomes for each treatment group. Statistically significant values are highlighted in grey; and

FIGS. 8A-8B show that D-leucine treatment after kainic acid-induced (25 mg/kg) seizure onset is not inferior to treatment with diazepam: (A) Mean seizure scores (±SEM) taken at 5-min intervals for 4 independent cohorts of mice treated with D-leucine (3 mg/kg), diazepam (10 mg/kg), a combination of D-leucine (3 mg/kg) and diazepam (10 mg/kg) or their respective vehicles (H₂O and PBS), 20 min after kainic acid (25 mg/kg); (B) Table showing seizure outcomes for each D-leucine treatment group in the kainic acid test. †P<0.0001 (ANOVA), P<0.001 H₂O+PBS vs DZP+H₂O, H₂O+PBS vs D-leu+PBS, H2O+PBS vs D-leu+DZP-leu (post-hoc Tukey); ††P<0.0001 (ANOVA), P<0.001 H₂O+PBS vs D-leu+PBS, H₂O+PBS vs D-leu+DZP, P<0.05 H2O+PBS vs DZP+H₂O (post-hoc Tukey); ‡P=0.37 (ANOVA). Rx, treatment time (D-leucine, diazepam, combination treatment or both).

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

The L-enantiomer of leucine is widely assumed to account for most of the biological effects of leucine. Selected D-amino acids (e.g., D-serine), however, exhibit biological activity. The presently disclosed subject matter demonstrates that D-amino acids protect against induced seizures and that D-amino acid therapy is useful for treating seizures. More particularly, the presently disclosed subject matter demonstrates that D-leucine, which is not incorporated into mammalian proteins and is not known to be involved in epilepsy, surprisingly protects against seizures more effectively than L-leucine (Hartman, et al., unpublished data). In addition, it has been found that another D-amino acid, D-serine, also protects against seizures. Further, it has also been found that D-amino acid oxidase inhibitors also protect against seizures.

Accordingly, in some embodiments, the presently disclosed subject matter provides methods using at least one D-amino acid for treating or preventing a seizure in a subject, the method comprising administering to the subject a therapeutically effective amount of at least one D-amino acid. In other embodiments, at least one D-amino acid is D-leucine or D-serine. In particular embodiments, the treating of a seizure reduces the frequency, severity, and/or duration of one or more seizures in the subject.

The D-amino acids of the presently disclosed subject matter include natural amino acids, such as histidine, alanine, isoleucine, arginine, leucine, asparagine, lysine, aspartic acid, methionine, cysteine, phenylalanine, glutamic acid, threonine, glutamine, tryptophan, valine, ornithine, proline, selenocysteine, serine, and tyrosine. Also, non-natural amino acids (i.e., compounds that do not occur in nature but that can be incorporated into a polypeptide chain) and/or amino acid analogs as are known in the art may alternatively be employed. Further, taurine (2-aminoethanesulfonic acid) may be considered an amino acid, and therefore, may be used in the presently disclosed methods. In addition, the amino acid may be modified, for example, by the addition of a chemical entity such as a carbohydrate group, a phosphate group, a farnesyl group, an isofarnesyl group, a fatty acid group, a linker for conjugation, functionalization, or other modification, and the like.

In addition, in some embodiments, the presently disclosed subject matter provides methods to treat or prevent a seizure in a subject by administering to the subject a therapeutically effective amount of at least one D-amino acid oxidase inhibitor, which is an inhibitor of D-amino acid degradation. It has been found that an inhibitor of D-amino acid metabolism, which increases endogenous levels of D-amino acids, also demonstrates protection against seizures. Examples of D-amino acid oxidase inhibitors include, but are not limited to, 5-chloro-benzo[d]isoxazol-3-ol (CBIO), 5-methylpyrazole-3-carboxylic acid (AS057278), 3-hydroxyquinolin-2(1H), compound 8 [4H-thieno[3,2-b]pyrrole-5-carboxylic acid], analogs thereof, and the like. In still other embodiments, at least one D-amino acid oxidase inhibitor is 5-chloro-benzo[d]isoxazol-3-ol (CBIO).

The presently disclosed methods are suitable for treating any subject that has had, is in the process of having, or is thought to be susceptible to having a seizure. The seizure may be caused by any disease, disorder, or dysfunction of the subject including, but not limited to, epilepsy, infantile spasms, Lennox Gastaut syndrome, a rapidly increasing fever (febrile seizure), an extremely low blood sugar level, for example, in a person afflicted with diabetes, damage to the brain from a stroke, brain surgery, or a head injury, congenital disorders, such as those caused by a genetic mutation or an inborn error of metabolism, withdrawal from alcohol, prescription medicine, or illegal drugs, an infection, such as meningitis or encephalitis, a brain tumor or structural defect in the brain, such as an aneurysm, or parasitic infections, such as tapeworm or toxoplasmosis. In some embodiments, the seizure is caused by a disease, disorder, or dysfunction selected from the group consisting of epilepsy, a rapidly increasing fever, low blood sugar, damage to the brain from a stroke, brain surgery, or a head injury, a congenital disorder, withdrawal from alcohol, prescription medicine, or illegal drugs, an infection, a brain tumor or structural defect in the brain, and a parasitic infection. In other embodiments, the seizure is caused by epilepsy.

Seizures vary in symptoms, frequency, severity, and duration from person to person and from episode to episode. Signs a subject is having a seizure include, but are not limited to, staring, eyelid fluttering, and abnormal sensory perceptions. In some occurrences, a subject may only have only slight shaking of a hand and does not lose consciousness. In other occurrences, a subject may become unconscious and have violent shaking of the entire body. In some other occurrences, shaking of the body, either mild or violent, may not occur with seizures. In further occurrences, a subject having a seizure may have symptoms before the seizure, such as seeing an aura or losing touch with their surroundings. In still further occurrences, the subject may be awake but may not respond to stimuli normally.

In some embodiments, the presently disclosed methods produce at least about a 10%, 15%, 20%, 25%, 30%, 35%, 40%, 45%, 50%, 55%, 60%, 65%, 70%, 75%, 80%, 85%, 90%, 95%, or even 100% reduction in frequency, severity, or duration of the seizures in a subject. In other embodiments, the frequency of seizures in a subject may remain approximately the same, but the severity and/or duration of the seizures may decrease. In still other embodiments, the subject is human. In further embodiments, the subject is non-human.

As used herein, the term “inhibit”, “inhibits”, “reduce” or “reduction” means to decrease, suppress, attenuate, diminish, arrest, or stabilize the development or progression of a disease, disorder, or condition, the activity of a biological pathway, or a biological activity, such as the frequency or type of seizures in a subject, e.g., by at least 10%, 20%, 30%, 40%, 50%, 60%, 70%, 80%, 90%, 95%, 98%, 99%, or even 100% compared to an untreated control subject, cell, biological pathway, or biological activity or compared to the symptoms seen in a subject before the subject is treated. As used herein, the term “decrease” means to inhibit, suppress, attenuate, diminish, arrest, or stabilize a symptom of a disease, disorder, or condition. It will be appreciated that, although not precluded, treating a disease, disorder or condition does not require that the disease, disorder, condition or symptoms associated therewith be completely eliminated.

In some embodiments, at least one D-amino acid or D-amino acid oxidase inhibitor is administered to the subject before the onset of a seizure, during a seizure, and/or after a seizure as a way to prevent further seizures. In other embodiments, at least one D-amino acid or D-amino acid oxidase inhibitor is administered to the subject prophylactically to prevent the occurrence of a seizure.

For use within the methods for treating seizures in a subject in need thereof, the D-amino acid or D-amino acid oxidase inhibitor described herein optionally may be administered in conjunction with other compounds or treatments useful in treating seizures or a disease associated with seizures, such as epilepsy. Accordingly, in some embodiments, the presently disclosed subject matter provides a method wherein at least one D-amino acid or D-amino acid oxidase inhibitor is administered in combination with another therapeutic agent. In other embodiments, the therapeutic agent is an agent known to prevent or treat seizures. Examples of therapeutic agents include, but are not limited to, diazepam (7-chloro-1,3-dihydro-1-methyl-5-phenyl-1,4-benzodiazepin-2(3H)-one; also known as Diastat® and Valium®), lorazepam, midazolam, clonazepam, propofol, phenytoin, valproate, levetiracetam, lacosamide, and the like. In still other embodiments, the therapeutic agent is diazepam. In further embodiments, the D-amino acid and D-amino acid oxidase inhibitor are administered together.

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. The terms “treat,” “treating,” “treatment,” and the like, as used herein can refer to curative therapy, prophylactic therapy, and preventative therapy. Accordingly, as used herein, “treating” means preventing or reducing the frequency, severity, and/or duration of seizures in a subject. The treatment, administration, or therapy can be consecutive or intermittent. Consecutive treatment, administration, or therapy refers to treatment on at least a daily basis without interruption in treatment by one or more days. Intermittent treatment or administration, or treatment or administration in an intermittent fashion, refers to treatment that is not consecutive, but rather cyclic in nature. Treatment according to the presently disclosed methods can result in complete relief or cure from a disease, disorder, or condition, or partial amelioration of one or more symptoms of the disease, disease, or condition, and can be temporary or permanent. The term “treatment” also is intended to encompass prophylaxis, therapy and cure.

As used herein, the terms “prevent,” “preventing,” “prevention,” “prophylactic treatment” and the like refer to reducing the probability of developing a disease, disorder, or condition in a subject, who does not have, but is at risk of or susceptible to developing a disease, disorder, or condition.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal (non-human) subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

### A. D-AMINO ACID AND D-AMINO ACID OXIDASE INHIBITOR COMPOSITIONS

The presently disclosed methods comprising at least one D-amino acid or D-amino acid oxidase inhibitor as described herein can be administered alone or in combination with one or more additional therapeutic agents, in admixture with a physiologically compatible carrier, which can be administered to a subject, for example, a human subject, for therapeutic or prophylactic treatment. As used herein, “physiologically compatible carrier” refers to a physiologically acceptable diluent including, but not limited to water, phosphate buffered saline, or saline, and, in some embodiments, includes another adjuvant.

Acceptable carriers, excipients, or stabilizers are nontoxic to recipients at the dosages and concentrations employed, and can include buffers, such as phosphate, citrate, and other organic acids; antioxidants including ascorbic acid, BHA, and BHT; low molecular weight (less than about 10 residues) polypeptides; proteins, such as serum albumin, gelatin or immunoglobulins; hydrophilic polymers, such as polyvinylpyrrolidone; amino acids, both L- and D-forms, such as glycine, glutamine, asparagine, arginine, or lysine; monosaccharides, disaccharides, and other carbohydrates including glucose, mannose, or dextrins; chelating agents, such as EDTA; sugar alcohols, such as mannitol or sorbitol; salt-forming counter-ions, such as sodium; and/or nonionic surfactants, such as Tween, Pluronics, or PEG.

In another embodiment, the D-amino acid or D-amino acid oxidase inhibitor of the presently disclosed subject matter further comprises another adjuvant. Additional adjuvants may include, but are not limited to, monophosphoryl lipid A (MPL); LTK63, dimethyl dioctadecyl-ammonium bromide (DDA), lipophilic quaternary ammonium salt-DDA, Trehalose dimycolate and synthetic derivatives, DDA-MPL, DDA-TDM, DDA-TDB, IC-31, aluminum salts, aluminum hydroxyide, aluminum phosphate, potassium aluminum phosphate, Montanide ISA-51, ISA-720, microparticles, immuno stimulatory complexes, liposomes, virosomes, virus-like particles, CpG oligonucleotides, cholera toxin, heat-labile toxin from E. coli, lipoproteins, dendritic cells, IL-12, GM-CSF, nanoparticles, a combination of soybean oil, emulsifying agents, and ethanol to form a nanoemulsion; ASO4, ZADAXIN, or combinations thereof.

Compositions to be used for in vivo administration must be sterile, which can be achieved by filtration through sterile filtration membranes, prior to or following lyophilization and reconstitution. Therapeutic compositions may be placed into a container having a sterile access port, for example, an intravenous solution bag or vial having a stopper pierceable by a hypodermic injection needle.

As described above, in certain embodiments, the presently disclosed subject matter also includes combination therapies. Additional therapeutic agents, which are normally administered to treat or prevent seizures, may be administered in combination with at least one D-amino acid or D-amino acid oxidase inhibitor as described herein. These additional agents may be administered separately, as part of a multiple dosage regimen, or may be part of a single dosage form, mixed together with at least one D-amino acid or D-amino acid oxidase inhibitor in a single composition.

By “in combination with” is meant the administration of at least one D-amino acid or D-amino acid oxidase inhibitor as described herein, with one or more therapeutic agents either simultaneously, sequentially, or a combination thereof. Therefore, a subject administered a combination of at least one D-amino acid or D-amino acid oxidase inhibitor and/or therapeutic agents, can receive the D-amino acid(s) or D-amino acid oxidase inhibitor(s) as described herein, and one or more therapeutic agents at the same time (i.e., simultaneously) or at different times (i.e., sequentially, in either order, on the same day or on different days), so long as the effect of the combination of both agents is achieved in the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where at least one D-amino acid or D-amino acid oxidase inhibitor and one or more therapeutic agents are administered simultaneously, they can be administered to the subject as separate pharmaceutical compositions, or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered agent is not diminished by the sequential, simultaneous or separate administration of the subsequent agent(s).

### B. DOSAGE AND MODE OF ADMINISTRATION

The presently disclosed compositions comprising at least one D-amino acid or D-amino acid oxidase inhibitor can be administered using a variety of methods known in the art depending on the subject and the particular disease, disorder, or condition being treated. The administering can be carried out by, for example, intravenous infusion; injection by intravenous, intraperitoneal, intracerebral, intramuscular, intraocular, intraarterial or intralesional routes; or topical or ocular application.

More particularly, as described herein, at least one D-amino acid or D-amino acid oxidase inhibitor can be administered to a subject for therapy by any suitable route of administration, including orally, nasally, transmucosally, ocularly, rectally, intravaginally, parenterally, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections, intracisternally, topically, as by powders, ointments or drops (including eyedrops), including buccally and sublingually, transdermally, through an inhalation spray, or other modes of delivery known in the art.

The phrases “systemic administration,” “administered systemically,” “peripheral administration” and “administered peripherally” as used herein mean the administration of at least one D-amino acid or D-amino acid oxidase inhibitor such that it enters the patient's system and, thus, is subject to metabolism and other like processes.

The phrases “parenteral administration” and “administered parenterally” as used herein mean modes of administration other than enteral and topical administration, usually by injection, and includes, without limitation, intravenous, intramuscular, intarterial, intrathecal, intracapsular, intraorbital, intraocular, intracardiac, intradermal, intraperitoneal, transtracheal, subcutaneous, subcuticular, intraarticular, subcapsular, subarachnoid, intraspinal and intrasternal injection and infusion.

Pharmaceutical compositions comprising at least one D-amino acid or D-amino acid oxidase inhibitor can be manufactured in a manner known in the art, e.g. by means of conventional mixing, dissolving, granulating, dragee-making, levitating, emulsifying, encapsulating, entrapping or lyophilizing processes.

More particularly, pharmaceutical compositions for oral use can be obtained through combination of at least one D-amino acid or D-amino acid oxidase inhibitor with a solid excipient, optionally grinding a resulting mixture, and processing the mixture of granules, after adding suitable auxiliaries, if desired, to obtain tablets or dragee cores. Suitable excipients include, but are not limited to, carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethyl cellulose; and gums including arabic and tragacanth; and proteins, such as gelatin and collagen; and polyvinylpyrrolidone (PVP:povidone). If desired, disintegrating or solubilizing agents, such as cross-linked polyvinyl pyrrolidone, agar, alginic acid, or a salt thereof, such as sodium alginate, also can be added to the compositions.

Dragee cores are provided with suitable coatings, such as concentrated sugar solutions, which also can contain gum arabic, talc, polyvinylpyrrolidone, carbopol gel, polyethylene glycol (PEG), and/or titanium dioxide, lacquer solutions, and suitable organic solvents or solvent mixtures. Dyestuffs or pigments can be added to the tablets or dragee coatings for product identification or to characterize the quantity of the D-amino acid or D-amino acid oxidase inhibitor compositions, e.g., dosage, or different combinations of doses.

Pharmaceutical compositions suitable for oral administration include push-fit capsules made of gelatin, as well as soft, sealed capsules made of gelatin and a coating, e.g., a plasticizer, such as glycerol or sorbitol. The push-fit capsules can contain active ingredients admixed with a filler or binder, such as lactose or starches, lubricants, such as talc or magnesium stearate, and, optionally, stabilizers. In soft capsules, at least one D-amino acid or D-amino acid oxidase inhibitor can be dissolved or suspended in suitable liquids, such as fatty oils, liquid paraffin, or liquid polyethylene glycols (PEGs), with or without stabilizers. Stabilizers can be added as warranted.

In some embodiments, at least one D-amino acid or D-amino acid oxidase inhibitor can be administered by rechargeable or biodegradable devices. For example, a variety of slow-release polymeric devices have been developed and tested in vivo for the controlled delivery of drugs, including proteinaceous biopharmaceuticals. Suitable examples of sustained release preparations include semipermeable polymer matrices in the form of shaped articles, e.g., films or microcapsules. Sustained release matrices include polyesters, hydrogels, polylactides (U.S. Pat. No. 3,773,919; EP 58,481), copolymers of L-glutamic acid and gamma ethyl-L-glutamate (Sidman et al., Biopolymers 22:547, 1983), poly (2-hydroxyethyl-methacrylate) (Langer et al., J. Biomed. Mater. Res. 15:167, 1981; Langer, Chem. Tech. 12:98, 1982), ethylene vinyl acetate (Langer et al., Id), or poly-D-(−)-3-hydroxybutyric acid (EP 133,988A).

Pharmaceutical compositions for parenteral administration include aqueous solutions of at least one D-amino acid or D-amino acid oxidase inhibitor. For injection, the presently disclosed pharmaceutical compositions can be formulated in aqueous solutions, for example, in some embodiments, in physiologically compatible buffers, such as Hank's solution, Ringer's solution, or physiologically buffered saline. Aqueous injection suspensions can contain substances that increase the viscosity of the suspension, such as sodium carboxymethyl cellulose, sorbitol, or dextran. Additionally, suspensions of the D-amino acid or D-amino acid oxidase inhibitor compositions include fatty oils, such as sesame oil, or synthetic fatty acid esters, such as ethyl oleate or triglycerides, or liposomes. Optionally, the suspension also can contain suitable stabilizers or agents that increase the solubility of the compositions.

For nasal or transmucosal administration generally, penetrants appropriate to the particular barrier to be permeated are used in the formulation. Such penetrants are generally known in the art.

For inhalation delivery, the agents of the disclosure also can be formulated by methods known to those of skill in the art, and may include, for example, but not limited to, examples of solubilizing, diluting, or dispersing substances, such as, saline, preservatives, such as benzyl alcohol, absorption promoters, and fluorocarbons.

Additional ingredients can be added to compositions for topical administration, as long as such ingredients are pharmaceutically acceptable and not deleterious to the epithelial cells or their function. Further, such additional ingredients should not adversely affect the epithelial penetration efficiency of the composition, and should not cause deterioration in the stability of the composition. For example, fragrances, opacifiers, antioxidants, gelling agents, stabilizers, surfactants, emollients, coloring agents, preservatives, buffering agents, and the like can be present. The pH of the presently disclosed topical composition can be adjusted to a physiologically acceptable range of from about 6.0 to about 9.0 by adding buffering agents thereto such that the composition is physiologically compatible with a subject's skin.

Regardless of the route of administration selected, the D-amino acid or D-amino acid oxidase inhibitor compositions are formulated into pharmaceutically acceptable dosage forms, such as described herein or by other conventional methods known to those of skill in the art.

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

Actual dosage levels of the active ingredients in the presently disclosed compositions can be varied so as to obtain an amount of the active ingredient that is effective to achieve the desired therapeutic response for a particular subject, composition, route of administration, and disease, disorder, or condition without being toxic to the subject. The selected dosage level will depend on a variety of factors including the activity of the particular composition comprising at least one D-amino acid or D-amino acid oxidase inhibitor, the route of administration, the time of administration, the duration of the treatment, other drugs and/or materials used in combination with the particular D-amino acid or D-amino acid oxidase inhibitor employed, the age, sex, weight, condition, general health and prior medical history of the patient being treated, and like factors well known in the medical arts.

A physician having ordinary skill in the art can readily determine and prescribe the effective amount of the D-amino acid or D-amino acid oxidase inhibitor composition required. Accordingly, the dosage range for administration will be adjusted by the physician as necessary.

Generally, doses of the D-amino acid or D-amino acid oxidase inhibitor will range from about 0.0001 to about 1000 mg per kilogram of body weight of the subject. In certain embodiments, the dosage is between about 1 μg/kg and about 500 mg/kg, more preferably between about 0.01 mg/kg and about 50 mg/kg. For example, in certain embodiments, a dose can be about 1, 5, 10, 15, 20, or 40 mg/kg.

### C. GENERAL DEFINITIONS

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1

### Materials and Methods

Animals:

Male NIH Swiss mice (NCI, Frederick, Md., U.S.A.) aged 3-4 weeks were acclimatized to the animal care facility for 1-5 days and housed four per cage. All mice were fed unrestricted normal rodent chow (Teklad Global 2018SX, Madison, Wis., U.S.A.). Only in experiments lasting 2 weeks, mice were fasted overnight before the D-leucine was administered in drinking water.

D-Amino Acid Administration:

At 5 weeks of age, mice were injected intraperitoneally with D-leucine 3 mg/kg (“low dose”) or 300 mg/kg (“high dose”) body weight (Sigma-Aldrich, St. Louis, Mo., U.S.A.) or given D-leucine in drinking water (1.5% w/v) for 13 days after an initial overnight fast. A similar intraperitoneal injection regimen was used for D-serine (300 mg/kg) (Sigma-Aldrich, St. Louis, Mo., U.S.A.).

Seizure Tests:

Each mouse was tested for seizures only once. Personnel performing seizure testing and assessments were blinded to treatment group assignments. Seizure tests were performed 3 h following a single dose of D-leucine, 15-25 min after the administration of kainic acid, or after 13 days of D-leucine exposure in drinking water.

6 Hz Test:

The 6 Hz test was administered using the same apparatus, stimulus frequency (6 Hz), pulse width (0.2 msec), and shock duration (3 sec) as described previously (Hartman et al., 2012). The primary outcome was the occurrence of seizures, defined as any abnormal activity of any duration, typically including clonus followed by immobility, facial muscle twitching, staring, automatisms including chewing and unilateral pawing, and a Straub tail.

Kainic Acid Test:

Kainic acid was injected intraperitoneally (22.5-25 mg kainic acid/kg mouse body mass, 5.3 mg/ml PBS, Tocris Bioscience, Ellisville, Mo., U.S.A.) as described previously (Hartman et al., 2012). Mice were observed continuously in plexiglass cages for the duration of the experiment. Seizure behaviors were scored for 2 h using a modified Racine scale (the highest score in a given 5-min block was used): 0, no seizure; 1, immobility; 2, forelimb and/or tail extension; 3, automatisms; 4, forelimb clonus, rearing, and/or falling; 5, repetition of stage 4; 6, tonic-clonic seizures; and 7, death (Hartman et al., 2010). As the mouse behaviors only occur intermittently, the highest seizure score achieved in a 5 min epoch was recorded.

Statistics:

Probit analyses (used in the 6 Hz test to determine the current where half the mice experienced any seizure behavior, or CC50, was performed using Minitab 16 (State College, Pa., U.S.A.). The level of significance in the 6 Hz test was 0.05. T-tests and one-way ANOVAs were performed using GraphPad Prism 4 (LaJolla, Calif., U.S.A.). In the kainic acid test, one-way ANOVAs were performed for multiple comparisons of 3 treatment groups. When 2 groups were compared in the kainic acid test, the level of significance was 0.01 (because 3-4 parameters were measured simultaneously, this represents a Bonferroni correction of a typical level of significance of 0.05).

### Example 2

### D-Amino Acid Pretreatment Protects Against Induced Seizures

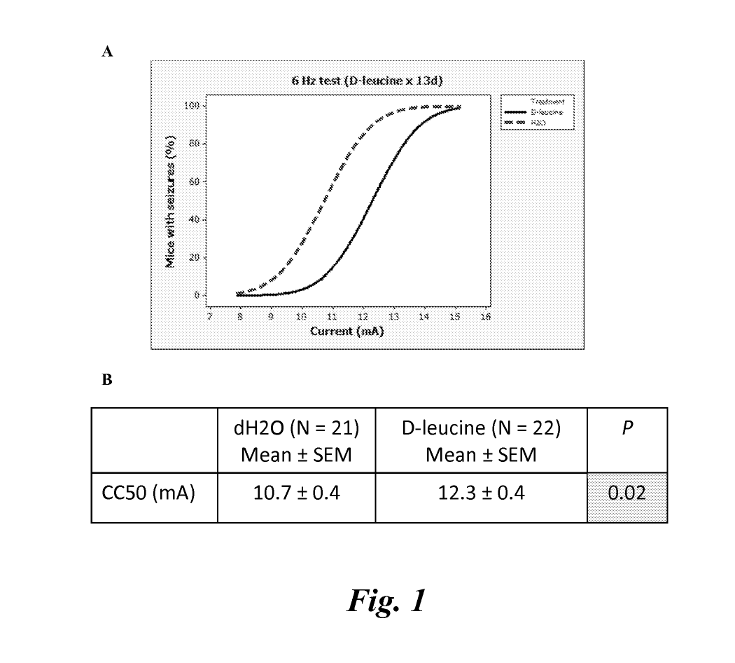

L-leucine protects against picrotoxin- and pentylenetetrazol-induced seizures in rats (Skeie et al., 1994; Dufour et al., 1999), but not against hexafluorodiethyl ether seizures (Gallagher, 1969). D-leucine may account for up to 0.5% of commercial L-leucine preparations (Sigma-Aldrich technical data). Thus, modest protection of L-leucine against seizures may be due to either isomer. Previous efforts used a paradigm modified from the Anticonvulsant Screening Project funded by the National Institute of Neurological Disease and Stroke (NINDS) (Smith et al., 2007). Because of its sensitivity in identifying antiseizure medicines, the 6 Hz test recently replaced the classical pentylenetetrazol test at the ASP (S. White, personal communication, December, 2012). Therefore, the effects of D-leucine were validated by employing the 6 Hz test. It has been shown previously that the 6 Hz test reliably reveals the antiseizure effects of the ketogenic diet in mice (Hartman et al., 2008). When administered in drinking water for 13 days, D-leucine increased the CC50 (i.e., current where half of mice had a convulsion) (FIGS. 1A-1B). A similar time frame (i.e. 11-13 days) was used to demonstrate the beneficial effects of the ketogenic diet and L-leucine in the 6 Hz test (Hartman et al., 2010; Hartman et al., 2008; Samala et al., 2008).

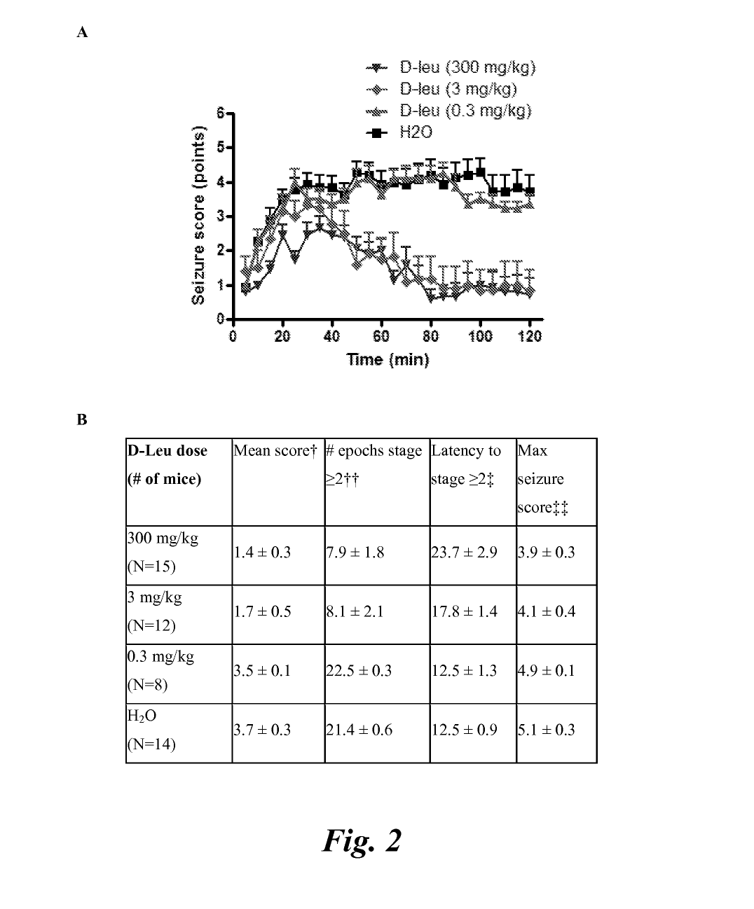

To demonstrate that the 6 Hz findings were not specific to only one method of seizure induction, the ability of D-leucine to protect against other convulsant stimuli using the kainic acid status epilepticus test, a widely-used model of temporal lobe epilepsy (i.e., the most common cause of epilepsy in adults in the US), was tested. At both high (300 mg/kg) and low (3 mg/kg) doses, D-leucine treatment prior to seizure induction protected against kainic acid-induced seizures, evidenced by decreased mean seizure scores and number of epochs with a seizure stage ≧2 (i.e., clinically obvious seizure activity) (FIGS. 2A-2B). At the high dose, D-leucine also prolonged the latency to onset of seizure stage ≧2 (FIGS. 2A-2B). The lowest dose tested (0.3 mg/kg) did not protect against seizures in this test. There was no difference between groups in the maximum seizure score. Thus, D-leucine decreases the duration of kainic acid-induced status epilepticus, although there was only a modest effect on decreasing the latency to onset of clinically obvious seizure activity. Thus, D-leucine protects against seizures induced via different convulsant stimuli.

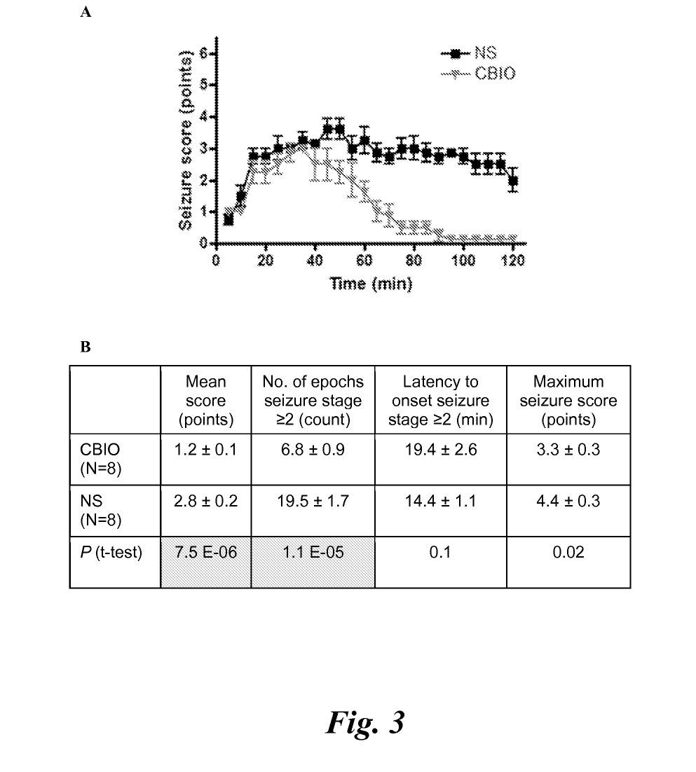

Using a different method of increasing levels of D-leucine, an inhibitor of its metabolism was tested via D-amino acid oxidase (CBIO, (5-chloro-benzo[d]isoxazol-3-01)) in the kainic acid test. The neurological relevance of D-amino acid oxidase is that genetic mutations in this enzyme have been linked to schizophrenia (Chumakov et al., 2002). CBIO, administered three hours prior to kainic acid, led to a decreased mean seizure score and number of epochs with a seizure stage ≧2 (FIGS. 3A-3B). There was no effect on latency to seizure stage ≧2 or maximum seizure score (FIGS. 3A-3B). Thus, both exogenous D-leucine administration and an inhibitor of its metabolism (i.e., increasing endogenous levels) demonstrate similar degrees of protection in the kainic acid test.

### Example 3

### D-Amino Acids Protect Against Ongoing Seizures after Initial Seizure Onset

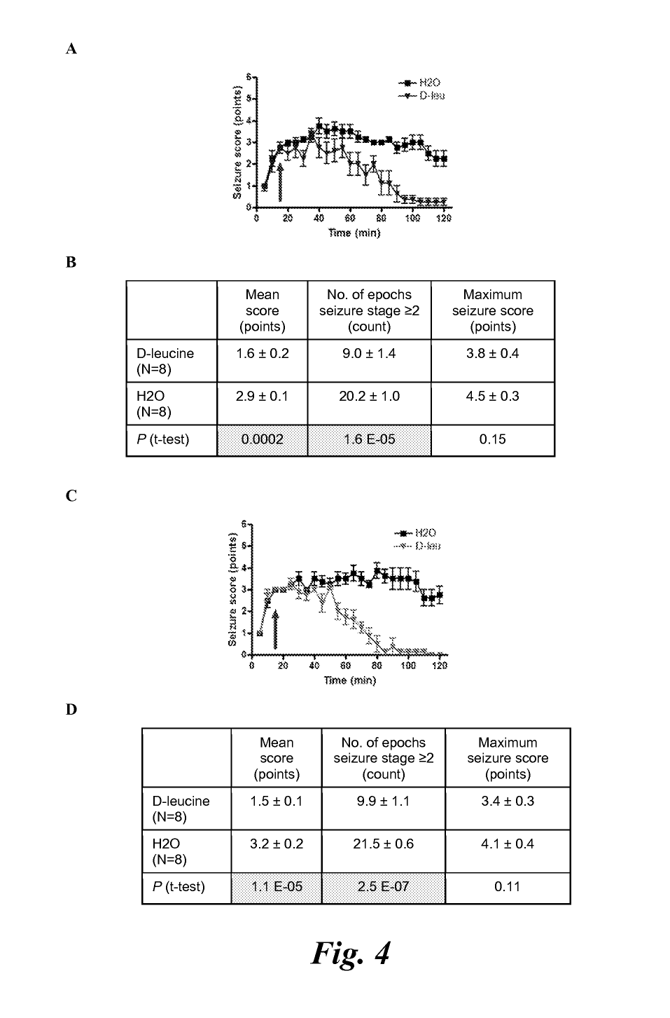

To further determine the translational and potential clinical relevance of these findings, high dose D-leucine was administered 15 min after kainic acid (the higher dose was chosen because it was more potent than the lower dose in the pretreatment studies described above). In this experiment, all mice had experienced at least one seizure with a score ≧3 when D-leucine was administered. D-leucine decreased the number of epochs with a seizure score ≧2 but had no effect on mean seizure score or maximum seizure score (but there was a trend for the latter) (FIGS. 4A-4B). Thus, D-leucine is effective in decreasing the duration of kainic acid-induced status epilepticus even when given after the onset of seizure activity.

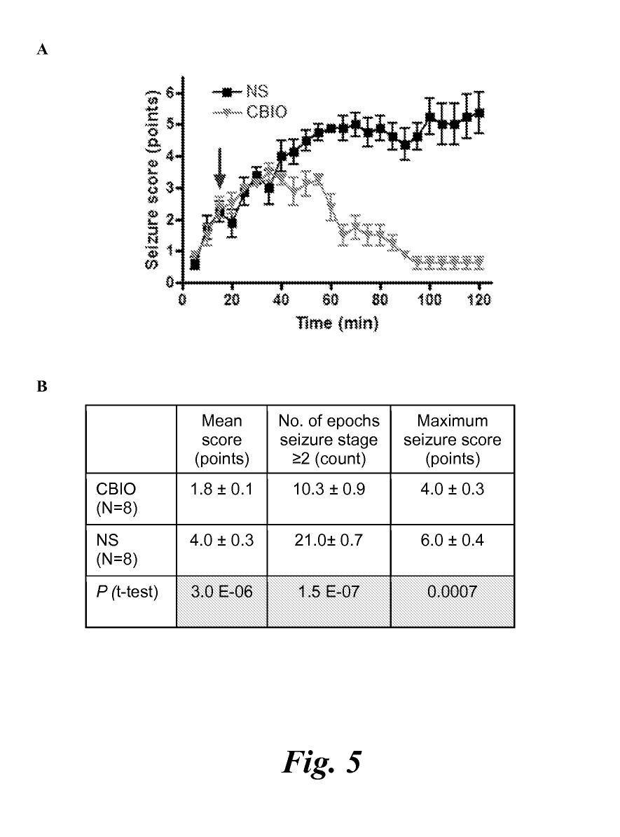

When administered 15 min after kainic acid, CBIO also led to a decrease in mean seizure score and number of epochs with a seizure stage ≧2, but had no effect on maximum seizure score (FIGS. 5A-5B). These data demonstrate that inhibiting D-leucine metabolism also protects against kainic acid-induced seizures after seizure onset.

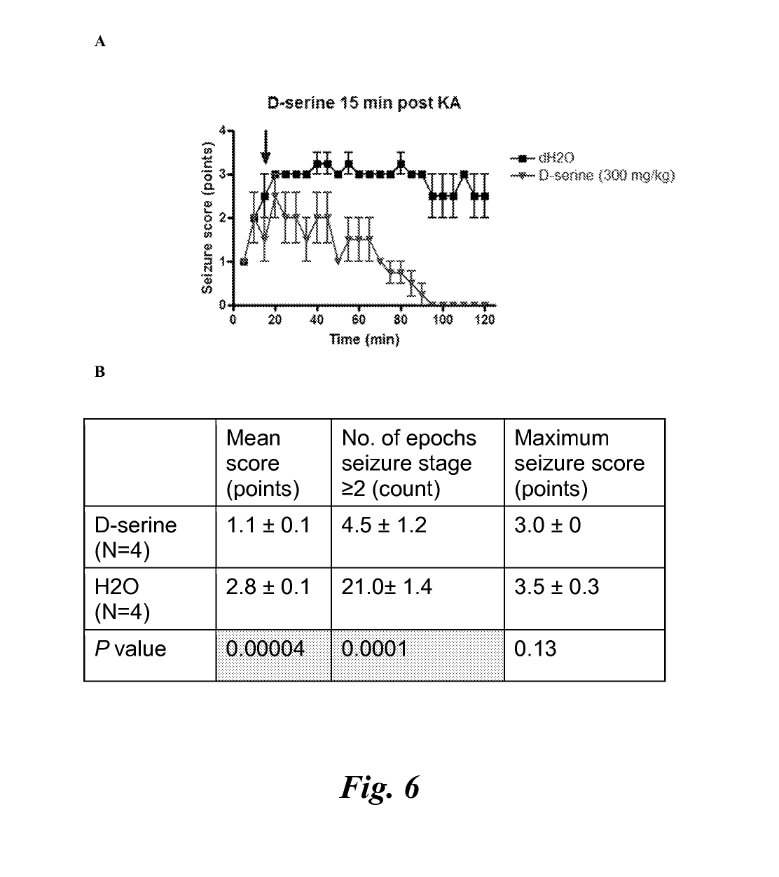

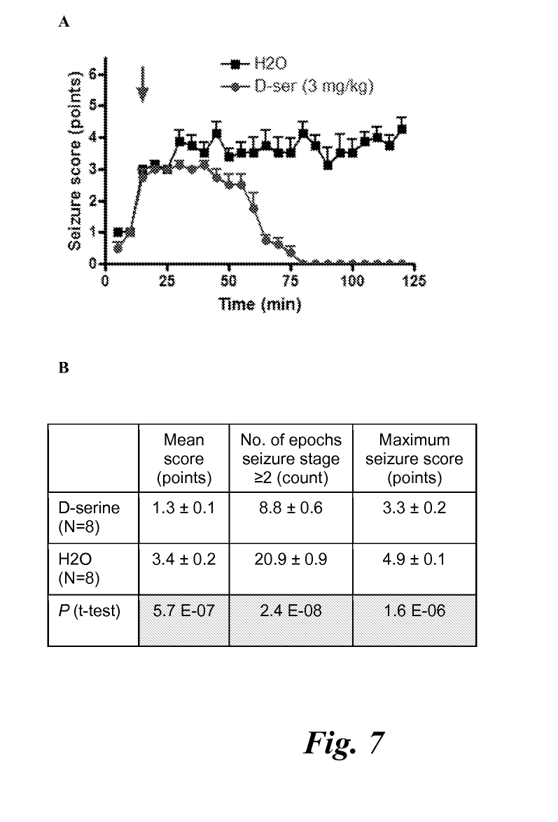

D-amino acid oxidase increases levels of other D-amino acids, including the most abundant D-amino acid in the brain, D-serine (Sacchi et al., 2012). Thus, whether D-serine administration protected against kainic acid-induced seizures was investigated. When administered 15 min after kainic acid, D-serine (300 mg/kg) led to a decrease in mean seizure score and number of epochs with a seizure stage ≧2 but there was no effect on maximum seizure score (FIGS. 6A-6B). In addition, when lower amounts of D-serine (3 mg/kg) were administered 15 min after kainic acid, D-serine led to a decrease in mean seizure score and number of epochs with a seizure stage ≧2 as well as a decrease in maximum seizure score (FIGS. 7A-7B). Thus, other D-amino acids protect against kainic acid-induced seizures, after seizure onset. These data suggest that inhibition of D-amino acid oxidase may be an effective strategy for stopping seizures.

### Example 4

### D-Amino Acid Combination Therapy

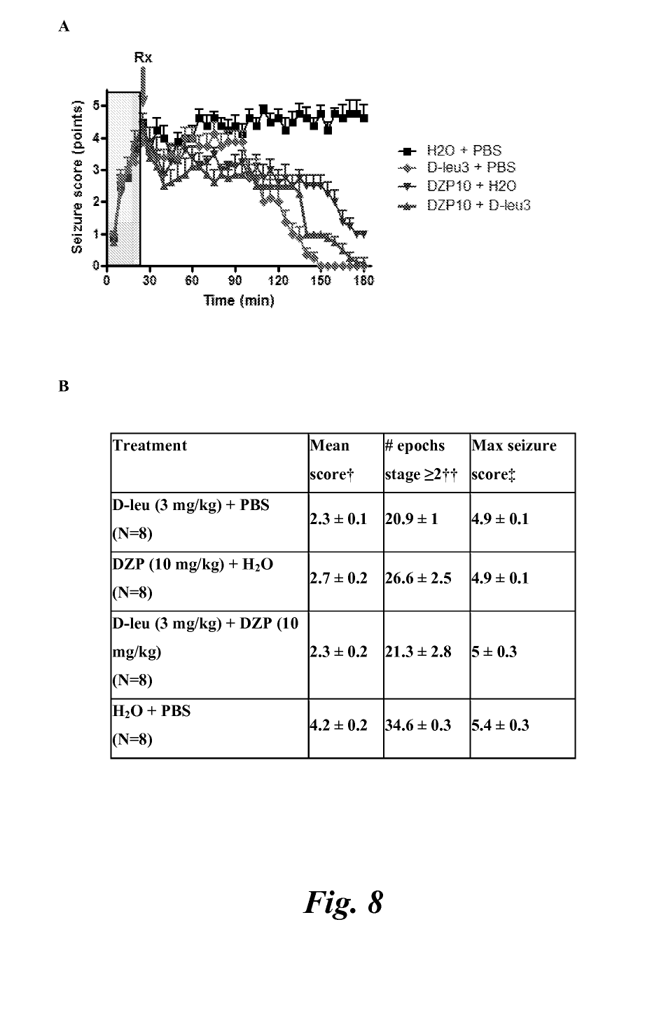

To determine if a combination therapy comprising a D-amino acid and another therapeutic agent was more effective in protecting against kainic acid-induced seizures after seizure onset than a D-amino acid alone, D-leucine and diazepam, a drug used to treat seizures, were administered together after induction by kainic acid (25 mg/kg in this experiment) in mice (FIGS. 8A-8B). Results showed that the combination of D-leucine and diazepam resulted in more effective protection against seizures. These results show that a combination therapy comprising a D-amino acid and another therapeutic agent can have a synergistic effect on the protection against seizures.

### Example 5

### Discussion

Data presented herein show that D-amino acids and D-amino acid oxidase inhibitors can treat or prevent seizures. D-leucine, which is not incorporated into mammalian proteins and is not known to be involved in epilepsy, protects against seizures more effectively than L-leucine (Hartman, et al., unpublished data). It has been further demonstrated that another D-amino acid, D-serine, decreases the duration of seizures in the kainic acid model of status epilepticus when given after a proconvulsant. D-leucine also protects against 6 Hz-induced seizures, demonstrating its utility in protecting against seizures induced by a variety of mechanisms. The inventors are unaware of any published work examining the effects of D-leucine or CBIO on seizures.

Commercial preparations of L-leucine may contain as much as 0.5% D-leucine (Sigma-Aldrich, technical data); thus, the potential activity of D-leucine also needs to be considered when interpreting data from prior studies of L-leucine in seizure models (Skeie et al., 1994; Dufour et al., 1999). The distinction between enantiomers may be important because leucine and other branched chain amino acids are under investigation for therapeutic use in patients lacking an enzyme in branched chain amino acid metabolism (Novarino et al., 2012).

In terms of the metabolism of D-amino acids, D-amino acids are oxidatively deaminated by the FAD-dependent enzyme, D-amino acid oxidase (DAAO), producing hydrogen peroxide and the respective imino acid; the latter is nonenzymatically hydrolyzed to ammonia and the corresponding α-ketoacid (Sacchi et al., 2012). However, the α-ketoacid of leucine (α-ketoisocaproic acid) is not active in the PTZ test (i.e., where leucine is protective), suggesting that α-ketoacids do not have an antiseizure effect (Dufour et al., 1999). DAAO is localized to neurons in the prefrontal cortex, hippocampus, and substantia nigra (note the anatomical overlap between D-leucine and DAAO in the hippocampus, the major seizure-producing structures in the brain), as well as other cells that have no direct defined role in seizure activity (e.g., Bergmann glia in the cerebellum) (Verrall et al., 2007). Mutations in DAAO have been linked genetically to schizophrenia, although the exact role played by the enzyme in this disorder remains to be elucidated (Chumakov et al., 2002). Inhibition of DAAO (i.e., with CBIO, FIGS. 3 and 5) may lead to decreased harmful ROS production (Sacchi et al., 2012). Without being bound to any one particular theory, because exogenous administration of two D-amino acids herein led to seizure control, it is believed that the data do not support decreased ROS levels as a primary mechanism of seizure protection in the kainic acid seizure test. Interestingly, neuroactive effects of CBIO were shown in one study demonstrating that it potentiated the antinociceptive effect of morphine in a rodent pain model (somewhat reminiscent of the antinociceptive effect of D-leucine noted previously in horses) (Gong et al., 2012).

Studies of D-amino acids involving D-serine are somewhat complicated by the fact that D-serine is metabolized by DAAO and serine racemase but the latter also is involved in the synthesis of D-serine (reviewed in Sachhi et al., 2012). Because the enzymatic synthesis and degradation of D-amino acids may lead to unexpected changes in their concentration (i.e., depending on which enzyme is more active in a given context, such as epilepsy), the levels of D-amino acids can be measured analytically (Rais et al., 2012). In addition, mouse knockout model of DAAO can be used to better understand the pharmacology of D-amino acids (Rais et al., 2012).

In terms of the potential mechanisms of action, without being bound to any one particular theory, it is believed that seizure protection at a low dose suggests that D-leucine may primarily act as a signaling molecule as the plasma membrane or other cellular membranes, rather than via bulk flow through amino acid transporters (as seen with other branched-chain and neutral amino acids (Yudkoff et al., 2007). Comparisons with D-serine are only of limited utility, as discussed herein below.

The use of D-serine in seizure tests has been examined but the results have been mixed. D-serine exerts weak but statistically significant protection against maximal electroshock-induced seizures; it potentiates the effect of clinical antiseizure medicines in the maximal electroshock test, and in a dose-dependent manner, increases after discharge thresholds in amygdala-kindled rats (Peterson, 1991; Loscher et al., 1994; Kalinichev et al., 2010). However, D-serine (given via the intracerebroventricular or intrathalamic routes) has no effect on spike wave discharges in GAERS (Genetic Absence Epilepsy Rats from Strasbourg) Wistar rats (Koerner et al., 1996).

In fact, there is some suggestion that D-serine enhances seizure activity. Serine racemase knockout mice are relatively protected against pentyletetrazol-induced seizures (Harai et al., 2012). Astrocytic levels of D-serine are increased in the pilocarpine model of temporal lobe epilepsy but the mechanism was not explored aside from immunohistochemical colocalization studies (Ryu et al., 2010). An earlier study indicated that D-serine levels (measured immunohistochemically, not biochemically) were elevated in degenerating GABAergic neurons in this model, although the reasons are unclear (Liu et al., 2009). D-serine decreases the antiseizure effect of the clinical medicine felbamate, the experimental compound L-687,414, and the opioid kappa-receptor agonist CI-977, among others (Singh et al., 1990; Tricklebank et al., 1994; De Sarro et al., 1994; White et al., 1995). However, rather than showing a direct proconvulsant effect of D-serine, the latter data were obtained to demonstrate the effect of these medicines at the glycine binding site on the NMDA receptor (i.e., by competition of D-serine at this site because D-serine is thought to be an endogenous ligand of this site).

In neurophysiology studies, D-serine inhibits hippocampal neuron kainic acid-induced AMPA receptor-mediated current (i.e., kainic acid and AMPA are ligands that bind to glutamatergic receptors that are pharmacologically distinct from NMDA receptors; kainic acid at modest to high doses activate both kainic acid and AMPA receptors) (Gong et al., 2007). In contrast, D-leucine was inactive in this assay, suggesting its mechanism of action is distinct from D-serine (Gong et al., 2007).

An alternative hypothesis is that D-leucine alters endogenous opioid levels, based on data showing D-leucine inhibits transport of enkephalins across the blood-brain barrier (Banks and Kastin, 1991). Opioids play a role in susceptibility to induced seizures, although their importance is unclear (Yajima et al., 2000).

In summary, the data show that the D-enantiomers of leucine and serine protect against different proconvulsants, including an effect after the onset of kainic acid-induced seizures. The data showed that D-leucine exerts antiseizure properties when administered prior to seizure testing in the 6 Hz and kainic acid tests. D-leucine, D-serine and CBIO, an inhibitor of D-amino acid metabolism also decrease seizure activity after seizure onset in the kainic acid test.

Specifically, in the kainic acid test, D-leucine pretreatment at both doses led to lower mean seizure scores and number of epochs spent in seizure stage ≧2. Treatment only at the high dose led to longer latency of onset to seizure stage ≧2. There was no difference between groups in the maximum seizure score. Treatment with either D-leucine or D-serine after kainic acid exposure led to decreased number of epochs spent in seizure stage ≧2 but only treatment with D-serine also led to decreased mean seizure score. Treatment with the D-amino acid oxidase inhibitor CBIO either before or after kainic acid injection also led to decreased mean seizure score and number of epochs spent in seizure stage ≧2, with no change in maximum seizure score.

Protection at a low dose suggests that D-leucine may act as a signaling molecule. Data from other groups support the hypothesis that D-leucine exerts its effect via pathways distinct from D-serine.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Banks W A, Kastin A J. Leucine modulates peptide transport system-1 across the blood-brain barrier at the stereospecific site within the central nervous system. J Pharm Pharmacol 1991; 43: 252-4.
Chumakov I, Blumenfeld M, Guerassimenko O, Cavarec L, Palicio M, Abderrahim H, Bougueleret L, Barry C, Tanaka H, La Rosa P, Puech A, Tahri N, Cohen-Akenine A, Delabrosse S, Lissarrague S, Picard F P, Maurice K, Essioux L, Millasseau P, Grel P, Debailleul V, Simon A M, Caterina D, Dufaure I, Malekzadeh K, Belova M, Luan J J, Bouillot M, Sambucy J L, Primas G, Saumier M, Boubkiri N, Martin-Saumier S, Nasroune M, Peixoto H, Delaye A, Pinchot V, Bastucci M, Guillou S, Chevillon M, Sainz-Fuertes R, Meguenni S, Aurich-Costa J, Cherif D, Gimalac A, Van Duijn C, Gauvreau D, Ouellette G, Fortier I, Raelson J, Sherbatich T, Riazanskaia N, Rogaev E, Raeymaekers P, Aerssens J, Konings F, Luyten W, Macciardi F, Sham P C, Straub R E, Weinberger D R, Cohen N, Cohen D. Genetic and physiological data implicating the new human gene G72 and the gene for D-amino acid oxidase in schizophrenia. Proc Natl Acad Sci USA 2002; 99: 13675-80.
Cota D, Proulx K, Smith K A, Kozma S C, Thomas G, Woods S C, Seeley R J. Hypothalamic mTOR signaling regulates food intake. Science 2006; 312: 927-30.
De Sarro G, Ongini E, Bertorelli R, Aguglia U, De Sarro A. Excitatory amino acid neurotransmission through both NMDA and non-NMDA receptors is involved in the anticonvulsant activity of felbamate in DBA/2 mice. Eur J Pharmacol 1994; 262: 11-9.
Dufour F, Nalecz K A, Nalecz M J, Nehlig A. Modulation of pentylenetetrazol-induced seizure activity by branched-chain amino acids and alpha-ketoisocaproate. Brain Res 1999; 815: 400-4.
Ekborg-Ott K H, Armstrong D W. Evaluation of the concentration and enantiomeric purity of selected free amino acids in fermented malt beverages (beers). Chirality 1996; 8: 49-57.
Gallagher B B. Amino acids and cerebral excitability. J Neurochem 1969; 16: 701-6.
Gong N, Wang Y C, Wang H L, Ma A N, Hashimoto K, Wang Y X. Interactions of the potent D-amino acid oxidase inhibitor CBIO with morphine in pain and tolerance to analgesia. Neuropharmacology 2012; 63: 460-8.
Gong X Q, Zabek R L, Bai D. D-Serine inhibits AMPA receptor-mediated current in rat hippocampal neurons. Can J Physiol Pharmacol 2007; 85: 546-55.
Hamase K, Homma H, Takigawa Y, Fukushima T, Santa T, Imai K. Regional distribution and postnatal changes of D-amino acids in rat brain. Biochim Biophys Acta 1997; 1334: 214-22.
Harai T, Inoue R, Fujita Y, Tanaka A, Horio M, Hashimoto K, Hongou K, Miyawaki T, Mori H. Decreased susceptibility to seizures induced by pentylenetetrazole in serine racemase knockout mice. Epilepsy Res 2012; 102: 180-7.
Hartman A L, Lyle M, Rogawski M A, Gasior M. Efficacy of the ketogenic diet in the 6-Hz seizure test. Epilepsia 2008; 49: 334-9.
Hartman A L, Santos P, Dolce A, Hardwick J M. The mTOR Inhibitor Rapamycin Has Limited Acute Anticonvulsant Effects in Mice. PLoS One 2012; 7: e45156.
Hartman A L, Stafstrom C E. Harnessing the power of metabolism for seizure prevention: focus on dietary treatments. Epilepsy Behav 2013; 26: 266-72.
Hartman A L, Zheng X, Bergbower E, Kennedy M, Hardwick J M. Seizure tests distinguish intermittent fasting from the ketogenic diet. Epilepsia 2010; 51: 1395-402.
Kalinichev M, Starr K R, Teague S, Bradford A M, Porter R A, Herdon H J. Glycine transporter 1 (GlyT1) inhibitors exhibit anticonvulsant properties in the rat maximal electroshock threshold (MEST) test. Brain Res 2010; 1331: 105-13.
Koerner C, Danober L, Boehrer A, Marescaux C, Vergnes M. Thalamic NMDA transmission in a genetic model of absence epilepsy in rats. Epilepsy Res 1996; 25: 11-9.
Liu Y H, Wang L, Wei L C, Huang Y G, Chen L W. Up-regulation of D-serine might induce GABAergic neuronal degeneration in the cerebral cortex and hippocampus in the mouse pilocarpine model of epilepsy. Neurochem Res 2009; 34: 1209-18.
Ljungberg M C, Sunnen C N, Lugo J N, Anderson A E, D'Arcangelo G. Rapamycin suppresses seizures and neuronal hypertrophy in a mouse model of cortical dysplasia. Dis Model Mech 2009; 2: 389-98.
Loscher W, Wlaz P, Rundfeldt C, Baran H, Honack D. Anticonvulsant effects of the glycine/NMDA receptor ligands D-cycloserine and D-serine but not R-(+)-HA-966 in amygdala-kindled rats. Br J Pharmacol 1994; 112: 97-106.
McKibbin L S, Cheng R S. Systemic d-phenylalanine and d-leucine for effective treatment of pain in the horse. Can Vet J 1982; 23: 39-40.
Novarino G, El-Fishawy P, Kayserili H, Meguid N A, Scott E M, Schroth J, Silhavy J L, Kara M, Khalil R O, Ben-Omran T, Ercan-Sencicek A G, Hashish A F, Sanders S J, Gupta A R, Hashem H S, Matern D, Gabriel S, Sweetman L, Rahimi Y, Harris R A, State M W, Gleeson J G. Mutations in BCKD-kinase lead to a potentially treatable form of autism with epilepsy. Science 2012; 338: 394-7.
Peterson S L. Anticonvulsant drug potentiation by glycine in maximal electroshock seizures is mimicked by D-serine and antagonized by 7-chlorokynurenic acid. Eur J Pharmacol 1991; 199: 341-8.
Rais R, Thomas A G, Wozniak K, Wu Y, Jaaro-Peled H, Sawa A, Strick C A, Engle S J, Brandon N J, Rojas C, Slusher B S, Tsukamoto T. Pharmacokinetics of oral D-serine in D-amino acid oxidase knockout mice. Drug Metab Dispos 2012; 40: 2067-73.
Ryu H J, Kim J E, Yeo S I, Kim D S, Kwon O S, Choi S Y, Kang T C. Potential roles of D-serine and serine racemase in experimental temporal lobe epilepsy. J Neurosci Res 2010; 88: 2469-82.
Sacchi S, Caldinelli L, Cappelletti P, Pollegioni L, Molla G. Structure-function relationships in human D-amino acid oxidase. Amino Acids 2012; 43: 1833-50.
Samala R, Willis S, Borges K. Anticonvulsant profile of a balanced ketogenic diet in acute mouse seizure models. Epilepsy Res 2008; 81: 119-27.
Sancak Y, Peterson T R, Shaul Y D, Lindquist R A, Thoreen C C, Bar-Peled L, Sabatini D M. The Rag GTPases bind raptor and mediate amino acid signaling to mTORC1. Science 2008; 320: 1496-501.
Singh L, Vass C A, Hunter J C, Woodruff G N, Hughes J. The anticonvulsant action of CI-977, a selective kappa-opioid receptor agonist: a possible involvement of the glycine/NMDA receptor complex. Eur J Pharmacol 1990; 191: 477-80.
Skeie B, Petersen A J, Manner T, Askanazi J, Steen P A. Effects of valine, leucine, isoleucine, and a balanced amino acid solution on the seizure threshold to picrotoxin in rats. Pharmacol Biochem Behav 1994; 48: 101-3.
Smith M, Wilcox K S, White H S. Discovery of antiepileptic drugs. Neurotherapeutics 2007; 4: 12-7.
Tricklebank M D, Bristow U, Hutson P H, Leeson P D, Rowley M, Saywell K, Singh L, Tattersall F D, Thorn L, Williams B J. The anticonvulsant and behavioural profile of L-687,414, a partial agonist acting at the glycine modulatory site on the N-methyl-D-aspartate (NMDA) receptor complex. Br J Pharmacol 1994; 113: 729-36.
Venall L, Walker M, Rawlings N, Benzel I, Kew J N, Harrison P J, Burnet P W. d-Amino acid oxidase and serine racemase in human brain: normal distribution and altered expression in schizophrenia. Eur J Neurosci 2007; 26: 1657-69.
White H S, Harmsworth W L, Sofia R D, Wolf H H. Felbamate modulates the strychnine-insensitive glycine receptor. Epilepsy Res 1995; 20: 41-8.
Yajima Y, Narita M, Takahashi-Nakano Y, Misawa M, Nagase H, Mizoguchi H, Tseng L F, Suzuki T. Effects of differential modulation of mu-, delta- and kappa-opioid systems on bicuculline-induced convulsions in the mouse. Brain Res 2000; 862: 120-6.
Yudkoff M, Daikhin Y, Melo T M, Nissim I, Sonnewald U. The ketogenic diet and brain metabolism of amino acids: relationship to the anticonvulsant effect. Annu Rev Nutr 2007; 27: 415-30.
Zeng L H, Rensing N R, Wong M. The mammalian target of rapamycin signaling pathway mediates epileptogenesis in a model of temporal lobe epilepsy. J Neurosci 2009; 29: 6964-72.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for treating or preventing a seizure in a subject, the method comprising administering to the subject a therapeutically effective amount of at least one D-amino acid.

2. The method of claim 1, wherein the at least one D-amino acid is D-leucine or D-serine.

3. The method of claim 1, wherein the at least one D-amino acid is administered in combination with another therapeutic agent.

4. The method of claim 3, wherein the therapeutic agent is diazepam.

5. The method of claim 1, wherein treating a seizure in a subject means reducing the frequency, severity, and/or duration of one or more seizures in the subject.

6. The method of claim 1, wherein the subject is human.

7. The method of claim 1, wherein the subject is non-human.

8. The method of claim 1, wherein the at least one D-amino acid is administered to the subject before an onset of a seizure, during a seizure, and/or after a seizure to prevent further seizures.

9. The method of claim 1, wherein the at least one D-amino acid is administered to the subject prophylactically to prevent the occurrence of a seizure.

10. The method of claim 1, wherein the seizure is caused by a disease, disorder, or dysfunction selected from the group consisting of epilepsy, a rapidly increasing fever, low blood sugar, damage to the brain from a stroke, brain surgery, or a head injury, a congenital disorder, withdrawal from alcohol, prescription medicine, or illegal drugs, an infection, a brain tumor or structural defect in the brain, and a parasitic infection.

11. The method of claim 1, wherein the seizure is caused by epilepsy.

12. A method for treating or preventing a seizure in a subject, the method comprising administering to the subject a therapeutically effective amount of at least one D-amino acid oxidase inhibitor.

13. The method of claim 12, wherein the at least one D-amino acid oxidase inhibitor is 5-chloro-benzo[d]isoxazol-3-ol (CBIO).

14. The method of claim 12, wherein treating a seizure in a subject means reducing the frequency, severity, and/or duration of one or more seizures in the subject.

15. The method of claim 12, wherein the subject is human.

16. The method of claim 12, wherein the subject is non-human.

17. The method of claim 12, wherein the at least one D-amino acid oxidase inhibitor is administered to the subject before an onset of a seizure, during a seizure, and/or after a seizure to prevent further seizures.

18. The method of claim 12, wherein the at least one D-amino acid oxidase inhibitor is administered to the subject prophylactically to prevent the occurrence of a seizure.

19. The method of claim 12, wherein the seizure is caused by a disease, disorder, or dysfunction selected from the group consisting of epilepsy, a rapidly increasing fever, low blood sugar, damage to the brain from a stroke, brain surgery, or a head injury, a congenital disorder, withdrawal from alcohol, prescription medicine, or illegal drugs, an infection, a brain tumor or structural defect in the brain, and a parasitic infection.

20. The method of claim 12, wherein the seizure is caused by epilepsy.
